(19) United States
(12) Patent Application Publication
(10) Pub. No.: US 2007/0232796 A1
(43) Pub. Date: Oct. 4, 2007

(54) Medical Solution, a Method for Producing Said Medical Solution and Use Thereof

(76) Inventors: Ola Carlsson, Lund (SE); Martin Erixon, Malmo (SE); Gunita Forsback, Loddekopinge (SE); Per Kjellstrand, Sodra Sandby (SE); Torbjorn Linden, Linderod (SE); Anders Wieslander, Lund (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/591,232

(22) PCT Filed: Feb. 22, 2005
(86) PCT No.: PCT/SE05/00238

§ 371 Date: May 7, 2007

Related U.S. Application Data
(60) Provisional application No. 60/549,225, filed on Mar. 1, 2004.

(30) Foreign Application Priority Data
Mar. 1, 2004 (SE) 0400522.9

(51) Int. Cl. A61K 31/7004; A61P 7/08

(52) U.S. Cl. 536/4.1

(57) ABSTRACT

The present invention relates to a medical solution comprising a first single solution containing glucose and/or glucose-like compound. According to the present invention said first single solution has a pH in the range of 1.8-2.6. The present invention further relates to a method for producing said medical solution as well as use thereof.

### TECHNICAL FIELD OF THE PRESENT INVENTION

The present invention relates to a medical solution comprising a first single solution containing glucose and/or glucose-like compounds, a method for producing said medical solution as well as use thereof.

### BACKGROUND OF THE INVENTION

Many medical solutions contain glucose and/or glucose-like compounds in different amounts are known. It is also known that glucose and/or glucose-like compounds give rise to problems during sterilization of medical solutions, see for example “Toxicity of peritoneal dialysis fluids on cultured fibroblasts L-929” by Anders Wieslander et al, Kidney International, Vol 40 (1991) pp 77-79.

During sterilization adding energy and/or during storage of solutions containing glucose and/or glucose-like compounds harmful components are formed. These components are formed by decomposition glucose and/or glucose-like compounds and are referred to as Glucose Degradation Products (GDPs).

During long-term peritoneal dialysis several problems arise as a consequence of the presence of GDPs in the Peritoneal Dialysis fluids (PD fluids).

Accordingly, new PD fluids have been developed with the aim to reduce the amount of GDPs.

The focused problem so far has been to reduce/minimize the total amount of GDPs in different solutions in order to keep the solution as biocompatible as possible.

The main part of these GDPs are however still unknown making the choice of a suitable marker for the reduction problematic.

One object of the present invention is thus to specifically optimize a medical solution in regard to biological reactivity and not only on the total amount of GDPs.

### SUMMARY OF THE INVENTION

The present invention relates to a medical solution comprising a first single solution containing glucose and/or glucose-like compound. According to the present invention said first single solution has a pH in the range of 1.8-2.6. The present invention further relates to a method for producing said medical solution as well as use thereof.

The inventors of the present invention have identified a new, highly reactive GDP named 3,4-dideoxyglucosone-3-ene (3,4-DGE), which has shown to be strongly connected to the cytotoxicity of PD fluids.

The present inventors have in several experiments found a good dose relation between 3,4-DGE and cytotoxicity, see FIG. 1.

Thus for the first time a medical fluid containing glucose and/or glucose-like compounds could be optimized with a clear and relevant focus, which is, minimizing the concentration of the highly toxic 3,4-DGE.

One advantage of the present invention is that it provides a medical solution having extremely low concentration of toxic 3,4-DGE.

Applied in Peritoneal Dialysis this medical solution likely preserve the peritoneal membrane of the patients during long-term dialysis.

In a preferred embodiment of the invention the pH of the first single solution is at least 1.8, preferably at least 2.0 and at most 2.6, preferably at most 2.5, most preferably at most 2.3.

In another preferred embodiment of the invention said medical solution further comprise a second single solution. This second single solution contains a buffer solution having such a pH and buffering capacity that when said first and second single solutions, up on use, are mixed to form a final solution, said final solution has a pH of 6.0-7.6.

In another preferred embodiment of the invention, said medical solution further contains one or more electrolytes, and the one or more electrolytes are preferably chosen from the group comprising ions of sodium, calcium, potassium, magnesium and/or chloride. Said one or more electrolytes is in one preferred embodiment arranged in said second single solution, but could also totally or to some extent be arranged in said first single solution.

In another preferred embodiment of the invention said medical solution further comprise a third single solution. Said third single solution also contains glucose and/or glucose-like compounds and has a pH of at least 1.8, preferably at least 2.0 and a pH of at most 2.6, preferably at most 2.5, most preferably at most 2.3. Said optional one or more electrolytes could also totally or to some extent be arranged in said third single solution.

In another preferred embodiment of the present invention said first and third single solutions contain different total amounts of glucose and/or glucose-like compounds. Up on use, said first or third single solution, individually or jointly is/are to be mixed with said second single solution to form a final solution. Said final solution has a pH in the range of 6.0-7.6.

According to one preferred embodiment of the invention, the different single solutions are provided in different compartments in a multicompartment bag before being mixed to the final solution.

As stated above the present invention also relates to a method for producing said medical solution. According to the method, said first single solution and optional second and third single solutions are provided in separate compartments. Thereafter said single solution(s) is(are) terminally sterilized.

In a preferred embodiment of the method according to the invention said terminal sterilization is heat sterilization and/or radiation sterilization. In another preferred embodiment of the invention, said terminal sterilization is heat sterilization at a temperature of at least 100° C., preferably at least 121° C.

In another embodiment of the method according to the present invention said first single solution and said second single solution, after terminal sterilization and up on use, are mixed to form a final solution.

In another embodiment of the method according to the present invention said second single solution and said third single solution, after terminal sterilization and up on use, are mixed to form a final solution.

In another embodiment of the method according to the present invention said first, second and third single solutions, after terminal sterilization and up on use, are mixed to form a final solution.

The present invention further relates to a multi-compartment bag comprising the medical solution according to above.

The present invention also relates to the use of the medical solution as disclosed above.

Additional objects, features, advantages and preferred embodiments of the present invention will become apparent from the following detailed description when taken in conjunction with the enclosed claims.

### DEFINITIONS

The term “medical solution” is intended to mean dialysis solutions for peritoneal dialysis, hemodialysis, hemodiafiltration, hemofiltration, for dialysis within renal intensive care, solutions for substitution or infusion normally containing glucose and/or glucose-like substances, and solutions for nutrition purposes.

The term “single solution” is intended to mean one solution kept isolated from other solutions up until use. The different single solutions could be used alone or mixed together with one or more single solutions up on use. Preferably, at least two single solutions are to be mixed up on use.

The term “glucose and/or glucose-like compound” is intended to mean compounds giving rise to glucose- or glucose polymer-derived toxins.

The term “buffer solution” is intended to mean solutions having a pH buffering capability to buffer the final solution into a pH range of 6.0-7.6.

The term “final solution” is intended to mean the solution which is ready to use and includes the required combination of different single solutions.

The term “electrolytes” is intended to mean the ions essential for the intended medical solution.

The term “multicompartment bag” is a bag divided into more than one compartment and that the content in the different compartment could be brought together and mixed before use.

The term “terminal sterilization” is intended to mean that the product is sterilized in its final package.

The terminal sterilization may include heat sterilization and/or radiation sterilization, but is preferably heat sterilization effected in an autoclave at a temperature of at least 100° C., preferably at least 121° C.

The term “up on use” is intended to mean as close as possible before the medical solution is used for it's specific purpose.

### BRIEF DESCRIPTION OF THE DRAWINGS

FIG. 1 is a diagram showing the relation ship between Inhibition of Cell Growth (ICG) and concentration of 3,4-DGE in a solution.

FIG. 2a is a diagram showing the results of different pH on 5-HMF measured at 0, 6 and 30 days (series 1, 2 and 3, respectively) after heat sterilization.

FIG. 2b is a diagram showing the results of different pH on 3-DG measured at 0, 6 and 30 days (series 1, 2 and 3, respectively) after heat sterilization.

FIG. 2c is a diagram showing the results of different pH on 3,4-DGE measured at 0, 6 and 30 days (series 1, 2 and 3, respectively) after heat sterilization.

FIG. 3 is a diagram showing the relative amounts of the 5-HMF, 3-DG and 3,4-DGE, respectively at different pH values after 30 days of incubation.

FIG. 4 is a diagram showing the sum of the 5-HMF, 3-DG and 3,4-DGE concentrations at different pH values after 30 days of incubation.

### DETAILED DESCRIPTION OF THE INVENTION

The highly toxic GDP, 3,4-DGE, has the two following cis and trans formulas:

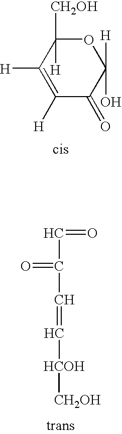

Other GDPs are for example acetaldehyde, formaldehyde, glyoxal, methylglyoxal, 3-deoxyglucosone (3-DG), 5-hydroxymethylfuraldehyd (5-HMF).

When glucose is exposed to sterilization adding energy, the following reaction takes place:

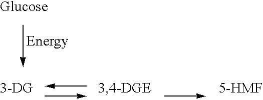

As shown above, 3-DG is in equilibrium with 3,4-DGE and 3,4-DGE is further transformed over to 5-HMF.

Extensive work has been done to optimize a medical solution containing glucose and/or glucose-like compounds in order to minimize the toxicity thereof.

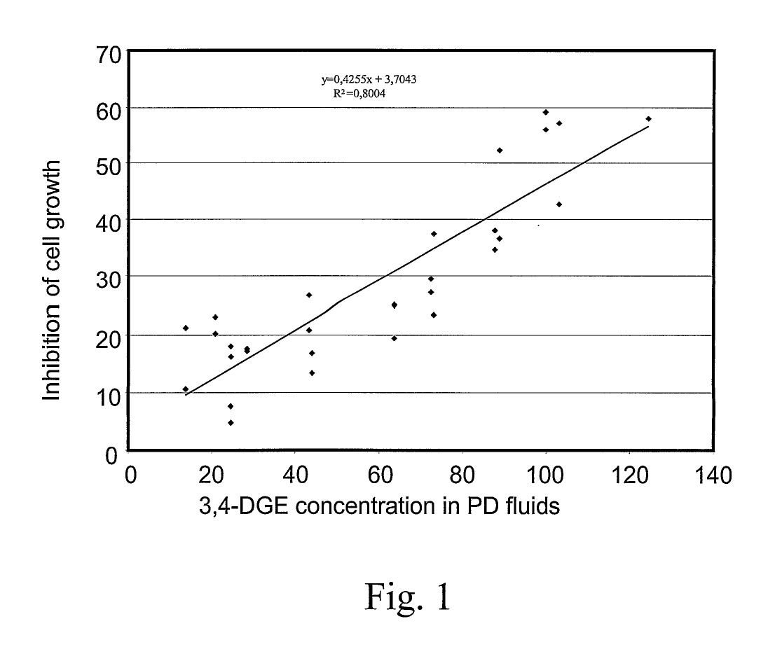

In order to investigate and come up with the present invention, the present inventors studied the relationship between the different GDPs and came to the conclusion that the GDPs interact according to the reaction formula given above. Further, the inventors investigated and found that 3,4-DGE is the mast toxic of the known GDPs and that there is a dose dependent relationship between the concentration of 3,4-DGE and the Inhibition of Cell Growth (ICG) used as a measure/marker for biological reactivity, see the enclosed FIG. 1.

Due to the correlation between 3-DG, 3,4-DGE and 5-HMF, the inventors chose to investigate the generated amounts of these GPDs in different solutions at different pH and after different storage time.

Solutions containing 50% by weight of glucose with different pH within the range of 1-4 were prepared. The different solutions were exposed for sterilization in form of heat sterilization in an autoclave at a temperature of 121° C. during 40 min.

The concentrations of 5-HMF, 3-DG and 3,4-DGE were measured after 0 days (serie 1), 6 days (serie 2) and 30 days (serie 3) after sterilization. During storage before the measurements at day 6 and 30, respectively, the solutions were kept at 40° C. to reflect the “worst storage scenario” possible.

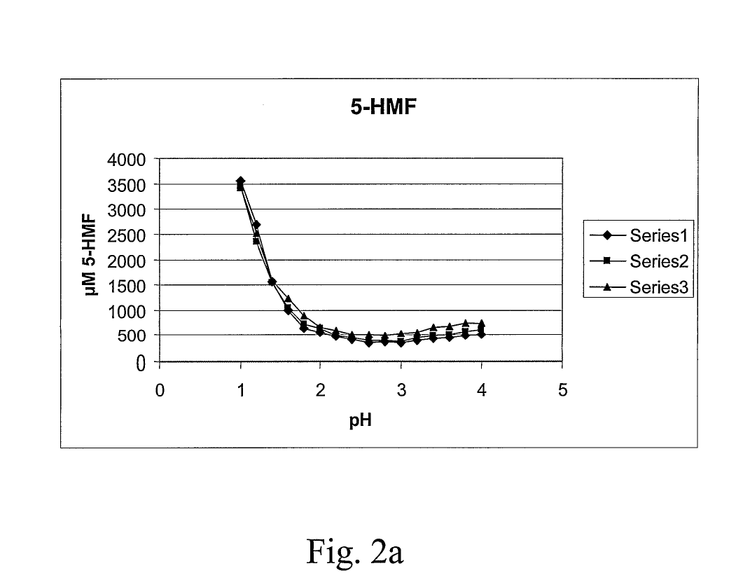

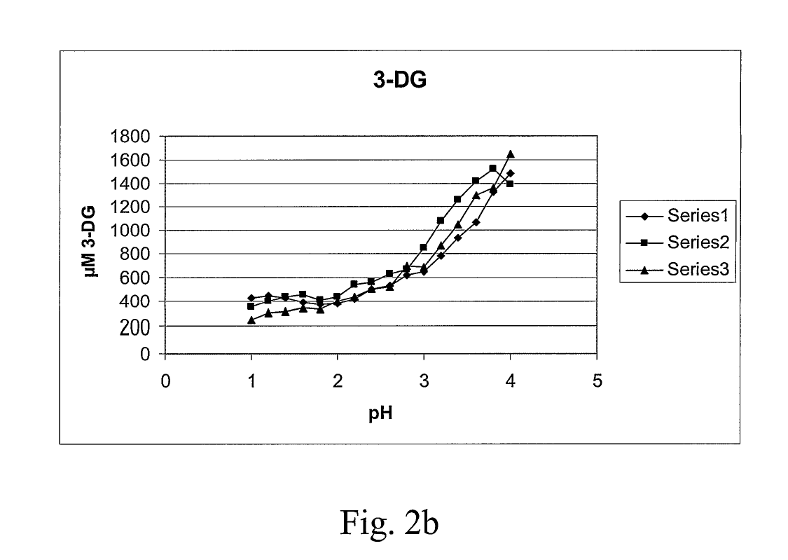

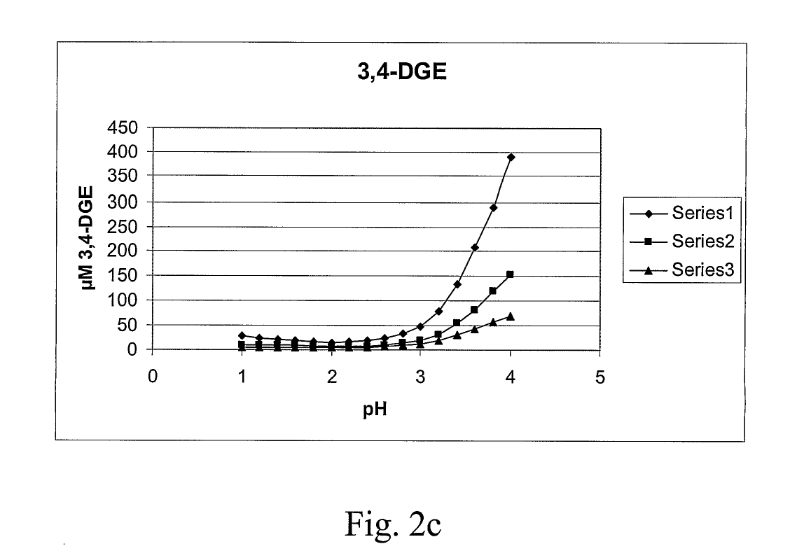

The results are shown in FIG. 2a-c for 5-HMF, 3-DG and 3,4-DGE, respectively.

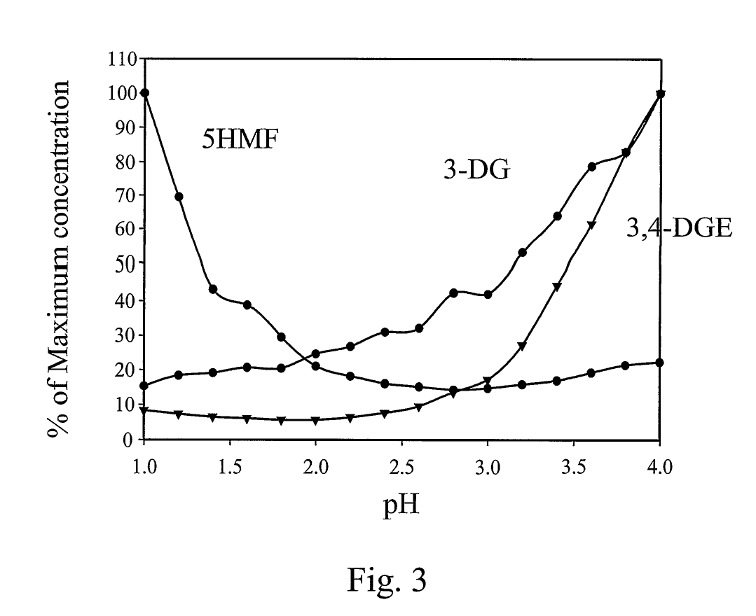

To be able to investigate the pH area which is the most optimal, the different results were put together in one diagram. To be able to weight the different GDPs after 30 days of storage in a correct way, the % of maximum concentration was plotted towards pH value. See FIG. 3 for the resulting diagram.

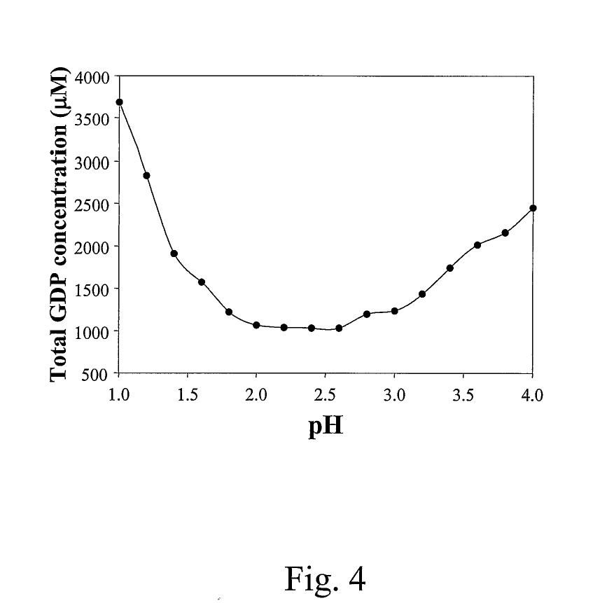

In FIG. 4 the sum of 3,4-DGE, 3-DG and 5 HMF concentrations after 30 days of storage has been plotted as a function of pH. It is, however, important to note that 3,4-DGE and 3-DG concentrations is more important than 5-HMF for the toxicity and a slight increase in the sum of the measured GDPs can be accepted in the lower range.

In the decision on how to choose the best pH range for the medical solution, it is not only important to keep the 3,4-DGE as low as possible. It is also important to look into the dynamics of the reaction formula for GDPs, as the reaction is an equlibrium between 3-DG and 3,4-DGE, while 5-HMF is an end product. This means that the 3-DG always could be changed into 3,4-DGE and vice versa. 5-HMF, on the other hand, is an end product, and after having been degraded to 5-HMF, there is no return to the most toxic GDP, namely, 3,4-DGE.

Knowing this, one quick solution to the problem seems to be to process the solution in such a way that all GDPs are reacted towards 5-HMF. However, 5-HMF still is a GDP and this GDP can participate in different polymerisation reactions and turn the medical solution yellow. It may possible cause not yet observed/described side-effects for the patient.

Accordingly, it is important to take all GDPs into consideration before deciding on the solution as such, and this is what the inventors of the present invention have done, and they have come up with the medical solution according to the claimed invention.

The medical solution according to the invention comprises a first single solution containing glucose and/or glucose-like compounds. This first single solution has a pH of at least 1.8, preferably at least 2.0 and a pH of at most 2.6, preferably at most 2.5, most preferably at most 2.3.

In one preferred embodiment the first single solution has a pH in the range of 1.8-2.6. In another preferred embodiment of the invention the first single solution has a pH in the range of 2.0-2.6. In another preferred embodiment of the invention the first single solution has a pH in the range of 1.8-2.5, and in even another preferred embodiment of the invention the first single solution has a pH in the range of 2.0-2.5.

In another preferred embodiment of the invention the first single solution has a pH in the range of 1.8-2.3, and in even another preferred embodiment of the invention the first single solution has a pH in the range of 2.0-2.3.

Besides optimizing the pH of the first single solution, the concentration of the glucose and/or glucose-like compounds also have an input, however, pH has the largest impact on the amount of produced GDPs. Preferred concentrations of glucose and/or glucose-like compounds in said first single solution is at least 10% by weight, preferably at least 20% by weight, and most preferably at least 40% by weight, based on the total weight of the first single solution.

In a preferred embodiment of the present invention the medical solution comprises a second single solution containing a buffer solution having such a pH and buffering capacity that when said first and second single solutions, up on use, are to be mixed to form a final solution, said final solution has a pH within the range of 6.0-7.6.

Preferably, the buffer solution comprises at least one of the following; bicarbonate, carbonate, acetate and lactate or combinations thereof.

If bicarbonate and/or carbonate is use either alone or in combination with any of the other suggested substances with buffering capability, a combination of bicarbonate and carbonate preferably is provided in such proportions that the partial pressure of carbon dioxide, CO₂, in the second single solution is of the same order of magnitude as the partial pressure of carbon dioxide, CO₂, in the atmosphere.

Using this buffer of a combination of bicarbonate and carbonate, an additional single solution comprising an acid and having a pH of 1.0-1.5, preferably 1.3 also is included. The second single solution comprising this combination of bicarbonate and carbonate preferably has a pH of 10.1-10.5, preferably 10.3. In this case said second single solution together with the additional single solution constitute the buffer solution.

In a preferred embodiment of the present invention the medical solution comprises a third single solution. This third single solution has the same preferred features as said first single solution.

In the preferred embodiment of the present invention the medical solution with said first, second and third single solution, said first and third single solutions could, according to a preferred embodiment of the invention, comprise different total amounts of glucose and/or glucose-like compounds. The different total amounts could be achieved by providing the same concentrations within said first and third single solutions, but providing different volumes thereof. The different total amounts could also be achieved by providing the same volume of said first and third single solutions, but providing different concentrations in said first single solution in comparison with said third single solution.

Having a preferred medical solution comprising said first, second and third single solutions, the user thereof could choose what concentration of glucose the user would like to have for a specific treatment. By combining said first and second single solutions to a final solution, the user gets a first specific concentration of glucose and/or glucose-like compounds, by combining said third and second single solutions to a final solution, the user gets a second specific concentration of glucose and/or glucose-like compounds, and by combining said first, second and third single solutions to a final solution, the user gets a third specific concentration of glucose and/or glucose-like compounds.

Accordingly, said first and third single solutions could, up on use, be mixed individually, i.e. either first or third single solution together with said second single solution, or joinly, i.e. both first and third single solutions together with said second single solution to form a final solution. Note that said final solution always have a pH within the range of 6.0-7.6, no matter which of the combinations above are used. The buffer solution in said second single solution have the capability to buffer said first and/or third solution(s) to a pH of 6.0-7.6 in the final solution.

Said single solutions could be provided in different compartments in a multi-compartment bag, and the mixing could be provided by having the different compartments sealingly coupled by frangible pins, which different pins could be broken in order to mix the content in optional compartments within the multicompartment bag. The mixing could further be provided by having a peal seal in between the different compartments, which peal seal could be pealed in order to mix the content in the different compartments. Two examples of multi-compartment bags are shown in FIGS. 5 and 6.

In another preferred embodiment of the invention the medical solution further contains one or more electrolytes. Preferably, the electolytes is one or more of the ions of sodium, calcium, potassium, magnesium and chloride.

The arrangement of electrolytes in the different compartments is dependent on the different electrolytes co-behavior with the other substances present in the single solutions, i.e. whether some sort of reaction could occur between the electrolyte(s) and the other substances present in a specific single solution(s). Usually, the electrolytes are contained in said second single solution. However, if bicarbonate is used as a buffer solution, the calcium ions preferably is provided in said first or third single solution. The reason for this is that calcium and bicarbonate together could cause precipition of calcium carbonate. However, calcium ions could be kept with bicarbonate under certain circumstances, such as in a specific pH range. This is for example disclosed in EP 0 437 274, which hereby is enclosed by reference.

In the method for producing the medical solution according to above, said first single solution and optional second and third single solutions are provided in separate compartments. Thereafter said single solution(s) is(are) terminally sterilized. Preferably the terminal sterilization is heat sterilization and/or radiation sterilization, (see also European Pharmacopoeia 1977 for a review of different sterilization techniques) In a preferred embodiment of the method according to the invention, the terminal sterilization is heat sterilization at a temperature of at least 100° C., preferably at least 121° C.

The sterilization time may vary depending on the sterilization temperature, the type of container and the contents therein to be sterilized.

The radiation sterilization may be either ionising or non-ionising sterilization. Examples of ionising sterilization are gamma and beta radiation. Example of non-ionizing radiation sterilization are UV radiation.

The medical solution according to the present invention has the advantage of having extremely low concentrations of the most toxic GDP, namely 3,4-DGE, and when used as a peritoneal dialysis solution it is likely to preserve the peritoneal membrane of the patients during long-term dialysis.

Below you will find different examples of solutions according to the present invention.

### EXAMPLES

By way of example, and not limitation, the following examples identify a variety of solutions made pursuant to an embodiment of the present invention.

### Example 1-3

Three-Compartment Bag with Lactate

Compartment 1 (First Single Solution):

| | Example 1 | Example 2 | Example 3 |
|---|---|---|---|
| Volume (l) | 0.062 | 0.062 | 0.062 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Third Single Solution):

| | Example 1 | Example 2 | Example 3 |
|---|---|---|---|
| Volume (l) | 0.103 | 0.103 | 0.103 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 3 (Second Single Solution):

| | Example 1 | Example 2 | Example 3 |
|---|---|---|---|
| Volume (l) | 1.96 | 1.96 | 1.96 |
| Na⁺ (mM) | 92.06 | 92.06 | 92.06 |
| Cl⁻ (mM) | 54.2 | 54.2 | 54.2 |
| Lactate (mM) | 42.11 | 42.11 | 42.11 |
| Ca²⁺ (mM) | 1.84 | 1.84 | 1.84 |
| Mg²⁺ (mM) | 0.27 | 0.27 | 0.27 |

Solution, Mixed and Ready for Use:

| | 1 + 3 | 2 + 3 | 1 + 2 + 3 |
|---|---|---|---|
| Example 1 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Lactate (mM) | 40.8 | 40.0 | 38.8 |
| Mg²⁺ (mM) | 0.26 | 0.26 | 0.26 |
| Ca²⁺ (mM) | 1.79 | 1.75 | 1.70 |
| Example 2 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Lactate (mM) | 40.8 | 40.0 | 38.8 |
| Mg²⁺ (mM) | 0.26 | 0.26 | 0.26 |
| Ca²⁺ (mM) | 1.79 | 1.75 | 1.70 |
| Example 3 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Lactate (mM) | 40.8 | 40.0 | 38.8 |
| Mg²⁺ (mM) | 0.26 | 0.26 | 0.26 |
| Ca²⁺ (mM) | 1.79 | 1.75 | 1.70 |

### Example 4-6

Three-Compartment Bag with Acetate

Compartment 1 (First Single Solution):

| | Example 4 | Example 5 | Example 6 |
|---|---|---|---|
| Volume (l) | 0.062 | 0.062 | 0.062 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Third Single Solution):

| | Example 4 | Example 5 | Example 6 |
|---|---|---|---|
| Volume (l) | 0.103 | 0.103 | 0.103 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 3 (Second Single Solution)

| | Example 4 | Example 5 | Example 6 |
|---|---|---|---|
| Volume (l) | 1.96 | 1.96 | 1.96 |
| Na⁺ (mM) | 92.06 | 92.06 | 92.06 |
| Cl⁻ (mM) | 54.2 | 54.2 | 54.2 |
| Acetate (mM) | 42.11 | 42.11 | 42.11 |
| Ca²⁺ (mM) | 1.84 | 1.84 | 1.84 |
| Mg²⁺ (mM) | 0.27 | 0.27 | 0.27 |

Solution, Mixed and Ready for Use:

| | 1 + 3 | 2 + 3 | 1 + 2 + 3 |
|---|---|---|---|
| Example 4 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 40.8 | 40.0 | 38.8 |
| Mg²⁺ (mM) | 0.26 | 0.26 | 0.26 |
| Ca²⁺ (mM) | 1.79 | 1.75 | 1.70 |
| Example 5 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 40.8 | 40.0 | 38.8 |
| Mg²⁺ (mM) | 0.26 | 0.26 | 0.26 |
| Ca²⁺ (mM) | 1.79 | 1.75 | 1.70 |
| Example 6 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 40.8 | 40.0 | 38.8 |
| Mg²⁺ (mM) | 0.26 | 0.26 | 0.26 |
| Ca²⁺ (mM) | 1.79 | 1.75 | 1.70 |

### Example 7-9

Three-Compartment Bag with Lactate+Acetate

Compartment 1 (First Single Solution):

| | Example 7 | Example 8 | Example 9 |
|---|---|---|---|
| Volume (l) | 0.062 | 0.062 | 0.062 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Third Single Solution):

| | Example 7 | Example 8 | Example 9 |
|---|---|---|---|
| Volume (l) | 0.103 | 0.103 | 0.103 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 3 (Second Single Solution):

| | Example 7 | Example 8 | Example 9 |
|---|---|---|---|
| Volume (l) | 1.96 | 1.96 | 1.96 |
| Na⁺ (mM) | 92.06 | 92.06 | 92.06 |
| Cl⁻ (mM) | 54.2 | 54.2 | 54.2 |
| Acetate (mM) | 12.11 | 12.11 | 12.11 |
| Lactate (mM) | 30.0 | 30.0 | 30.0 |
| Mg²⁺ (mM) | 1.84 | 1.84 | 1.84 |
| Ca²⁺ (mM) | 0.27 | 0.27 | 0.27 |

Solution, Mixed and Ready for Use:

| | 1 + 3 | 2 + 3 | 1 + 2 + 3 |
|---|---|---|---|
| Example 7 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 11.7 | 11.5 | 11.2 |
| Lactate (mM) | 29.1 | 28.5 | 27.7 |
| Mg²⁺ (mM) | 0.26 | 0.26 | 0.26 |
| Ca²⁺ (mM) | 1.79 | 1.75 | 1.70 |
| Example 8 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 11.7 | 11.5 | 11.2 |
| Lactate (mM) | 29.1 | 28.5 | 27.7 |
| Mg²⁺ (mM) | 0.26 | 0.26 | 0.26 |
| Ca²⁺ (mM) | 1.79 | 1.75 | 1.70 |
| Example 9 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 11.7 | 11.5 | 11.2 |
| Lactate (mM) | 29.1 | 28.5 | 27.7 |
| Mg²⁺ (mM) | 0.26 | 0.26 | 0.26 |
| Ca²⁺ (mM) | 1.79 | 1.75 | 1.70 |

### Examples 10-12

Three-Compartment Bag with Lactate+Bicarbonate

Compartment 1 (First Single Solution):

| | Example 10 | Example 11 | Example 12 |
|---|---|---|---|
| Volume (l) | 0.062 | 0.062 | 0.062 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Third Single Solution):

| | Example 10 | Example 11 | Example 12 |
|---|---|---|---|
| Volume (l) | 0.103 | 0.103 | 0.103 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 3 (Second Single Solution):

| | Example 10 | Example 11 | Example 12 |
|---|---|---|---|
| Volume (l) | 1.96 | 1.96 | 1.96 |
| Na⁺ (mM) | 92.06 | 92.06 | 92.06 |
| Cl⁻ (mM) | 54.2 | 54.2 | 96.28 |
| Bicarbonate (mM) | 12.11 | 12.11 | 12.11 |
| Lactate (mM) | 30.0 | 30.0 | 30.0 |
| Mg²⁺ (mM) | 1.84 | 1.84 | 1.84 |
| Ca²⁺ (mM) | 0.27 | 0.27 | 0.27 |

Solution, Mixed and Ready for Use:

| | 1 + 3 | 2 + 3 | 1 + 2 + 3 |
|---|---|---|---|
| Example 11 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Bicarbonate (mM) | 11.7 | 11.5 | 11.2 |
| Lactate (mM) | 29.1 | 28.5 | 27.7 |
| Mg²⁺ (mM) | 0.26 | 0.26 | 0.26 |
| Ca²⁺ (mM) | 1.79 | 1.75 | 1.70 |
| Example 11 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Bicarbonate (mM) | 11.7 | 11.5 | 11.2 |
| Lactate (mM) | 29.1 | 28.5 | 27.7 |
| Mg²⁺ (mM) | 0.26 | 0.26 | 0.26 |
| Ca²⁺ (mM) | 1.79 | 1.75 | 1.70 |
| Example 12 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Bicarbonate (mM) | 11.7 | 11.5 | 11.2 |
| Lactate (mM) | 29.1 | 28.5 | 27.7 |
| Mg²⁺ (mM) | 0.26 | 0.26 | 0.26 |
| Ca²⁺ (mM) | 1.79 | 1.75 | 1.70 |

### Example 13-15

Three-Compartment Bag with Acetate+Bicarbonate

Compartment 1 (First Single Solution):

| | Example 13 | Example 14 | Example 15 |
|---|---|---|---|
| Volume (l) | 0.062 | 0.062 | 0.062 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Third Single Solution):

| | Example 13 | Example 14 | Example 15 |
|---|---|---|---|
| Volume (l) | 0.103 | 0.103 | 0.103 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 3 (Second Single Solution):

| | Example 13 | Example 14 | Example 15 |
|---|---|---|---|
| Volume (l) | 1.96 | 1.96 | 1.96 |
| Na⁺ (mM) | 92.06 | 92.06 | 92.06 |
| Cl⁻ (mM) | 54.2 | 54.2 | 54.2 |
| Acetate (mM) | 30.0 | 30.0 | 30.0 |
| Bicarbonate (mM) | 12.11 | 12.11 | 12.11 |
| Mg²⁺ (mM) | 1.84 | 1.84 | 1.84 |
| Ca²⁺ (mM) | 0.27 | 0.27 | 0.27 |

Solution, Mixed and Ready for Use:

| | 1 + 3 | 2 + 3 | 1 + 2 + 3 |
|---|---|---|---|
| Example 13 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 29.1 | 28.5 | 27.7 |
| Bicarbonate (mM) | 11.7 | 11.5 | 11.2 |
| Mg²⁺ (mM) | 0.26 | 0.26 | 0.26 |
| Ca²⁺ (mM) | 1.79 | 1.75 | 1.70 |
| Example 14 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 29.1 | 28.5 | 27.7 |
| Bicarbonate (mM) | 11.7 | 11.5 | 11.2 |
| Mg²⁺ (mM) | 0.26 | 0.26 | 0.26 |
| Ca²⁺ (mM) | 1.79 | 1.75 | 1.70 |
| Example 15 | | | |
| Volume (l) | 2.022 | 2.063 | 2.125 |
| Glucose (g/l) | 15.33 | 24.96 | 38.82 |
| Na⁺ (mM) | 132.9 | 132.1 | 130.9 |
| Cl⁻ (mM) | 55.3 | 56.1 | 57.1 |
| Acetate (mM) | 29.1 | 28.5 | 27.7 |
| Bicarbonate (mM) | 11.7 | 11.5 | 11.2 |
| Mg²⁺ (mM) | 0.26 | 0.26 | 0.26 |
| Ca²⁺ (mM) | 1.79 | 1.75 | 1.70 |

### Example 16-18

Two-Compartment Bag with Lactate, 2.5% Glucose

Compartment 1 (First Single Solution):

| | Example 16 | Example 17 | Example 18 |
|---|---|---|---|
| Volume (l) | 0.100 | 0.100 | 0.100 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Second Single Solution):

| | Example 16 | Example 17 | Example 18 |
|---|---|---|---|
| Volume (l) | 1.9 | 1.9 | 1.9 |
| Na⁺ (mM) | 134.1 | 134.1 | 134.1 |
| Cl⁻ (mM) | 91.4 | 91.4 | 91.4 |
| Lactate (mM) | 42.11 | 42.11 | 42.11 |
| Mg²⁺ (mM) | 0.26 | 0.26 | 0.26 |
| Ca²⁺ (mM) | 1.84 | 1.84 | 1.84 |

Solution, Mixed and Ready for Use:

| | 1 + 2 |
|---|---|
| Example 16 | |
| Volume (l) | 2 |
| Glucose (g/l) | 25 |
| Na⁺ (mM) | 132 |
| Cl⁻ (mM) | 96 |
| Lactate (mM) | 40 |
| Mg²⁺ (mM) | 0.25 |
| Ca²⁺ (mM) | 1.75 |
| Example 17 | |
| Volume (l) | 2 |
| Glucose (g/l) | 25 |
| Na⁺ (mM) | 132 |
| Cl⁻ (mM) | 96 |
| Lactate (mM) | 40 |
| Mg²⁺ (mM) | 0.25 |
| Ca²⁺ (mM) | 1.75 |
| Example 18 | |
| Volume (l) | 2 |
| Glucose (g/l) | 25 |
| Na⁺ (mM) | 132 |
| Cl⁻ (mM) | 96 |
| Lactate (mM) | 40 |
| Mg²⁺ (mM) | 0.25 |
| Ca²⁺ (mM) | 1.75 |

### Example 19-21

Two-Compartment Bag with Acetate+Bicarbonate

Compartment 1 (First Single Solution):

| | Example 19 | Example 20 | Example 21 |
|---|---|---|---|
| Volume (l) | 0.100 | 0.100 | 0.100 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 2 (Second Single Solution):

| | Example 19 | Example 20 | Example 21 |
|---|---|---|---|
| Volume (l) | 1.9 | 1.9 | 1.9 |
| Na⁺ (mM) | 134.1 | 134.1 | 134.1 |
| Cl⁻ (mM) | 91.4 | 91.4 | 91.4 |
| Bicarbonate (mM) | 12.11 | 12.11 | 12.11 |
| Acetate (mM) | 30.0 | 30.0 | 30.0 |
| Mg²⁺ (mM) | 0.26 | 0.26 | 0.26 |
| Ca²⁺ (mM) | 1.84 | 1.84 | 1.84 |

Solution, Mixed and Ready for Use:

| | 1 + 3 |
|---|---|
| Example 19 | |
| Volume (l) | 1.9 |
| Glucose (g/l) | 25 |
| Na⁺ (mM) | 132 |
| Cl⁻ (mM) | 96 |
| Bicarbonate (mM) | 11.5 |
| Acetate (mM) | 28.5 |
| Mg²⁺ (mM) | 1.75 |
| Ca²⁺ (mM) | 0.25 |
| Example 20 | |
| Volume (l) | 1.9 |
| Glucose (g/l) | 25 |
| Na⁺ (mM) | 132 |
| Cl⁻ (mM) | 96 |
| Bicarbonate (mM) | 11.5 |
| Acetate (mM) | 28.5 |
| Mg²⁺ (mM) | 1.75 |
| Ca²⁺ (mM) | 0.25 |
| Example 21 | |
| Volume (l) | 1.9 |
| Glucose (g/l) | 25 |
| Na⁺ (mM) | 132 |
| Cl⁻ (mM) | 96 |
| Bicarbonate (mM) | 11.5 |
| Acetate (mM) | 28.5 |
| Mg²⁺ (mM) | 1.75 |
| Ca²⁺ (mM) | 0.25 |

### Example 22-24

Three-Compartment Bag with Lactate+Bicarbonate, Equal Volumes Glucose Concentrate

Compartment 1 (First Single Solution):

| | Example 22 | Example 23 | Example 24 |
|---|---|---|---|
| Volume (l) | 0.103 | 0.103 | 0.103 |
| Glucose (g/l) | 300 | 300 | 300 |
| Na⁺ (mM) | 55.4 | 55.4 | 55.4 |
| Cl⁻ (mM) | 55.4 | 55.4 | 55.4 |
| pH | 1.8 | 2.1 | 2.5 |

Compartment 2 (Third Single Solution):

| | Example 22 | Example 23 | Example 24 |
|---|---|---|---|
| Volume (l) | 0.103 | 0.103 | 0.103 |
| Glucose (g/l) | 500 | 500 | 500 |
| Na⁺ (mM) | 92.06 | 92.06 | 92.06 |
| Cl⁻ (mM) | 92.06 | 92.06 | 92.06 |
| pH | 1.8 | 2.1 | 2.5 |

Compartment 3 (Second Single Solution):

| | Example 22 | Example 23 | Example 24 |
|---|---|---|---|
| Volume (l) | 1.96 | 1.96 | 1.96 |
| Na⁺ (mM) | 92.06 | 92.06 | 92.06 |
| Cl⁻ (mM) | 54.2 | 54.2 | 54.2 |
| Lactate (mM) | 30.0 | 30.0 | 30.0 |
| Bicarbonate (mM) | 12.11 | 12.11 | 12.11 |
| Mg²⁺ (mM) | 0.27 | 0.27 | 0.27 |
| Ca²⁺ (mM) | 1.84 | 1.84 | 1.84 |

Solution, Mixed and Ready for Use:

| | 1 + 3 | 2 + 3 | 1 + 2 + 3 |
|---|---|---|---|
| Example 22 | | | |
| Volume (l) | 2.063 | 2.063 | 2.166 |
| Glucose (g/l) | 14.99 | 24.96 | 38.04 |
| Na⁺ (mM) | 90.23 | 92.06 | 90.36 |
| Cl⁻ (mM) | 54.2 | 56.1 | 56.0 |
| Lactate (mM) | 29.1 | 28.5 | 27.7 |
| Bicarbonate (mM) | 11.7 | 11.5 | 11.2 |
| Mg²⁺ (mM) | 0.25 | 0.25 | 0.24 |
| Ca²⁺ (mM) | 1.75 | 1.75 | 1.74 |
| Example 23 | | | |
| Volume (l) | 2.063 | 2.063 | 2.166 |
| Glucose (g/l) | 14.99 | 24.96 | 38.04 |
| Na⁺ (mM) | 90.23 | 92.06 | 90.36 |
| Cl⁻ (mM) | 54.2 | 56.1 | 56.0 |
| Lactate (mM) | 29.1 | 28.5 | 27.7 |
| Bicarbonate (mM) | 11.7 | 11.5 | 11.2 |
| Mg²⁺ (mM) | 0.25 | 0.25 | 0.24 |
| Ca²⁺ (mM) | 1.75 | 1.75 | 1.74 |
| Example 24 | | | |
| Volume (l) | 2.063 | 2.063 | 2.166 |
| Glucose (g/l) | 14.99 | 24.96 | 38.04 |
| Na⁺ (mM) | 90.23 | 92.06 | 90.36 |
| Cl⁻ (mM) | 54.2 | 56.1 | 56.0 |
| Lactate (mM) | 29.1 | 28.5 | 27.7 |
| Bicarbonate (mM) | 11.7 | 11.5 | 11.2 |
| Mg²⁺ (mM) | 0.25 | 0.25 | 0.24 |
| Ca²⁺ (mM) | 1.75 | 1.75 | 1.74 |

### Example 25-28

Four-Compartment Bags, Containing Bicarbonate:

Compartment 1 (Second Single Solution):

| | Example | | Example | | Example | | Example | |
|---|---|---|---|---|---|---|---|---|
| | 25 | | 26 | | 27 | | 28 | |
| NaHCO₃ | 95.5 | mM | 112 | mM | 139 | mM | 133 | mM |
| Na₂CO₃ | 304.5 | mM | 258 | mM | 661 | mM | 607 | mM |
| pH | 10.3 | | 10.3 | | 10.3 | | 10.3 | |
| Volume | 0.196 | l | 0.196 | l | 0.098 | l | 0.098 | l |

Compartment 2 (Additional Single Solution):

| | Example | | Example | | Example | | Example | |
|---|---|---|---|---|---|---|---|---|
| | 25 | | 26 | | 27 | | 28 | |
| HCl | 38.74 | mM | 38.74 | mM | 39.19 | mM | 39.19 | mM |
| NaCl | 78.88 | mM | 78.88 | mM | 71.94 | mM | 71.94 | mM |
| CaCl₂*2H₂O | 1.99 | mM | 1.99 | mM | 1.88 | mM | 1.88 | mM |
| MgCl₂*6 H₂O | 0.57 | mM | 0.57 | mM | 0.54 | mM | 0.54 | mM |
| Lactate | — | | 3.41 | mM | — | | 3.22 | mM |
| pH | 1.3 | | 1.3 | | 1.3 | | 1.3 | |
| Volume | 1.764 | l | 1.764 | l | 1.862 | l | 1.862 | l |

Compartment 3 (First Single Solution):

| | Example | | Example | | Example | | Example | |
|---|---|---|---|---|---|---|---|---|
| | 25 | | 26 | | 27 | | 28 | |
| Glucose | 500 | g/l | 500 | g/l mM | 500 | g/l mM | 500 | g/l |
| pH | 2.0 | | 2.0 | | 2.0 | | 2.0 | |
| Volume | 0.062 | l | 0.062 | l | 0.062 | l | 0.062 | l |

Compartment 4 (Third Single Solution):

| | Example | | Example | | Example | | Example | |
|---|---|---|---|---|---|---|---|---|
| | 25 | | 26 | | 27 | | 28 | |
| Glucose | 500 | g/l | 500 | g/l mM | 500 | g/l mM | 500 | g/l |
| pH | 2-2.6 | | 2-2.6 | | 2-2.6 | | 2-2.6 | |
| Volume | 0.103 | l | 0.103 | l | 0.103 | l | 0.103 | l |

Solution, Mixed and Ready for Use:

| Comparment | Compartment | Compartment |
|---|---|---|
| 1 + 2 + 3 | 1 + 2 + 4 | 1 + 2 + 3 + 4 |

| Example 25 | | | | | | |
|---|---|---|---|---|---|---|
| Volume | 2.022 | l | 2.063 | l | 2.125 | l |
| pH | 7.0 | | 7.0 | | 7.0 | |
| Cl⁻ | 107.8 | mM | 105.6 | mM | 102.5 | mM |
| Na⁺ | 137.1 | mM | 134.4 | mM | 130.5 | mM |
| Ca⁺ | 1.74 | mM | 1.70 | mM | 1.65 | mM |
| Mg⁺ | 0.50 | mM | 0.49 | mM | 0.47 | mM |
| HCO₃⁻ | 38.8 | mM | 38.0 | mM | 36.9 | mM |
| Glucose | 15.3 | g/l | 25.0 | g/l | 38.8 | g/l |
| Example 26 | | | | | | |
| Volume | 2.022 | l | 2.063 | l | 2.125 | l |
| pH | 7.0 | | 7.0 | | 7.0 | |
| Cl⁻ | 107.8 | mM | 105.6 | mM | 102.5 | mM |
| Na⁺ | 135.6 | mM | 132.9 | mM | 129.1 | mM |
| Ca⁺ | 1.74 | mM | 1.70 | mM | 1.65 | mM |
| Mg⁺ | 0.50 | mM | 0.49 | mM | 0.47 | mM |
| HCO₃⁻ | 35.9 | mM | 35.2 | mM | 34.1 | mM |
| Glucose | 15.3 | g/l | 25.0 | g/l | 38.8 | g/l |
| Lactate | 2.98 | mM | 2.92 | mM | 2.83 | mM |
| Example 27 | | | | | | |
| Volume | 2.022 | l | 2.063 | l | 2.125 | l |
| pH | 7.0 | | 7.0 | | 7.0 | |
| Cl⁻ | 113.2 | mM | 110.9 | mM | 107.7 | mM |
| Na⁺ | 137.1 | mM | 134.3 | mM | 130.5 | mM |
| Ca⁺ | 1.73 | mM | 1.70 | mM | 1.65 | mM |
| Mg⁺ | 0.50 | mM | 0.49 | mM | 0.47 | mM |
| HCO₃⁻ | 38.8 | mM | 38.0 | mM | 36.9 | mM |
| Glucose | 15.3 | g/l | 25.0 | g/l | 38.8 | g/l |
| Example 28 | | | | | | |
| Volume | 2.022 | l | 2.063 | l | 2.125 | l |
| pH | 7.0 | | 7.0 | | 7.0 | |
| Cl⁻ | 113.2 | mM | 110.9 | mM | 107.7 | mM |
| Na⁺ | 137.5 | mM | 134.7 | mM | 130.8 | mM |
| Ca⁺ | 1.73 | mM | 1.70 | mM | 1.65 | mM |
| Mg⁺ | 0.5 | mM | 0.49 | mM | 0.47 | mM |
| HCO₃⁻ | 38.8 | mM | 38.0 | mM | 36.9 | mM |
| Glucose | 15.3 | g/l | 25.0 | g/l | 38.8 | g/l |
| Lactate | 2.97 | mM | 2.91 | mM | 2.82 | mM |

In summary, based on the above results, the inventors concluded that a highly biocompatible medical solution containing glucose and/or glucose-like compounds can be prepared, provided that the solution containing glucose and/or glucose-like compounds has a pH within the range of 1.8-2.6.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A medical solution comprising a first single solution containing glucose and/or a glucose-like compound, wherein said first single solution has a pH in the range of 1.8-2.6.

2. A medical solution according to claim 1, wherein said first single solution has a pH in the range of 2.0-2.6.

3. A medical solution according to claim 1, wherein said first single solution has a pH in the range of 1.8-2.5.

4. A medical solution according to claim 1, wherein said first single solution has a pH in the range of 2.0-2.5.

5. A medical solution according to claim 1, wherein said first single solution has a pH in the range of 1.8-2.3.

6. A medical solution according to claim 1, wherein said first single solution has a pH in the range of 2.0-2.3.

7. A medical solution according to claim 1, wherein the medical solution further comprises a second single solution containing a buffer solution having a pH and buffering capacity such that upon mixing of said first and second single solutions to form a final solution, said final solution has a pH of 6.0-7.6.

8. A medical solution according to claim 1 or 7, wherein the medical solution further contains one or more electrolytes.

9. A medical solution according to claim 8, wherein said one or more electrolytes comprise one or more of the ions of sodium, calcium, potassium, magnesium or chloride.

10. A medical solution according to claim 1, further comprising a second single solution having one or more electrolytes.

11. A medical solution according to claim 8, wherein the one or more electrolytes are arranged in said first single solution.

12. A medical solution according to claim 7, wherein the medical solution further comprises a third single solution, said third single solution containing glucose and/or glucose-like compounds and having a pH of at least 1.8 at most 2.6.

13. A medical solution according to claim 12, wherein one or more electrolytes are arranged in said third single solution.

14. A medical solution according to claim 12 or claim 13, wherein said first and third single solutions contain different total amounts of glucose and/or glucose-like compounds, and said first and third single solutions when mixed with said second single solution, either individually or jointly, form a final solution having a pH in the range of 6.0-7.6.

15. A medical solution according to claim 7, wherein the first and second single solutions are provided in different compartments in a multi-compartment bag before being mixed to form the final solution.

16. A method for producing a medical solution according to claim 1, said method comprising:
providing said first single solution in a compartment; and thereafter
terminally sterilizing said single solution.

17. A method according to claim 16, wherein said step of terminally sterilizing comprises heat sterilization and/or radiation sterilization.

18. A method according to claim 16, wherein said step of terminally sterilizing comprises heat sterilization at a temperature of at least 100° C.

19. A method according to claim 25, wherein said first single solution and said second single solution, after terminal sterilization, are mixed to form a final solution.

20. A method according to claim 26, wherein said second single solution and said third single solution, after terminal sterilization, are mixed to form a final solution.

21. A method according to claim 26, wherein said first single solution, said third single solution, and said second single solution, after terminal sterilization, are mixed to form a final solution.

22. A multi-compartment bag comprising the medical solution according to one of claims 1, 7, and 12.

23. (canceled)

24. A medical solution according to claim 12, wherein the first, second, and third single solutions are provided in different compartments in a multi-compartment bag before being mixed to form the final solution.

25. A method for producing a medical solution according to claim 7, said method comprising:
providing said first and second single solutions in separate compartments; and
thereafter
terminally sterilizing said first and second single solutions.

26. A method for producing a medical solution according to claim 12, said method comprising:
providing said first, second, and third single solutions in separate compartments;
and thereafter
terminally sterilizing said first, second, and third single solutions.

27. A method according to claim 25 or 26, wherein said step of terminally sterilizing comprises heat sterilization and/or radiation sterilization.

28. A method according to claim 25 or 26, wherein said step of terminally sterilizing comprises heat sterilization at a temperature of at least 100° C.
